(19) United States
(12) Patent Application Publication
(10) Pub. No.: US 2014/0276217 A1
(43) Pub. Date: Sep. 18, 2014

(54) FLUID SAMPLING APPARATUS AND METHOD

(71) Applicants: Lars Otto LIEPOLD, St. Louis Park, MN (US); Darren Patrick LIEPOLD, St. Louis Park, MN (US)

(72) Inventors: Lars Otto LIEPOLD, St. Louis Park, MN (US); Darren Patrick LIEPOLD, St. Louis Park, MN (US)

(21) Appl. No.: 14/216,761

(22) Filed: Mar. 17, 2014

Related U.S. Application Data
(60) Provisional application No. 61/798,535, filed on Mar. 15, 2013.

(51) Int. Cl. A61B 5/15; A61B 19/00

(52) U.S. Cl. CPC A61B 5/150358; A61B 5/150305; A61B 19/2203
USPC 600/573

(57) ABSTRACT

The present invention relates generally to a sampling apparatus and a method for bodily fluids, and, more specifically, a blood sampling apparatus and a method for using the same. A blood collection system includes a housing having at least one open end, and an absorbent material positioned within the housing. The absorbent material has at least one end positioned proximate the at least one open end of the housing. A method of collecting blood samples includes positioning an absorbent material near a source of blood, holding the absorbent material with a housing, the absorbent material absorbing a volume of blood, and moving the housing and absorbent material to a position where the absorbent material can dry.

### CROSS-REFERENCE TO RELATED APPLICATION

This application claims the benefit of U.S. provisional application No. 61/798,535, filed on 15 Mar. 2013, the contents of which are incorporated herein by reference. A claim of priority is made.

### TECHNICAL FIELD

Various embodiments described herein relate to a sampling apparatus and a method for bodily fluids, and, more specifically, a blood sampling apparatus and a method for using the same.

### BACKGROUND

For many years, methods of testing for diseases, drugs and other antigens in humans have years been done using blood samples. These samples are collected in laboratories at the request of physicians. Generally, these tests require that blood is drawn by a trained phlebotomist is sent to a laboratory. Drawn blood is left in a test tube. These are relatively inefficient from the standpoint of shipping. Test tubes are also somewhat fragile. Shipping blood is also very time sensitive. The serum component of the blood, comprising a predominance of immunoglobulins, that contain antibodies to the disease or disease state in question, is tested using a variety of available test kits to assist in the diagnosis of various diseases including infectious diseases, cardiovascular diseases, cancers and many others. Such samples can also be tested for the presence of non-disease analytes such as metals, minerals, DNA, bacteria and organic molecules among others.

Another type of test is dried blood testing. This is called dry bloodspot sampling. In 1963, Robert Guthrie developed dried blood spot sampling for neonatal screening of metabolic disorders. Dried-blood-spot-cards are sometimes referred to as “Guthrie Cards”. Dry bloodspot sampling has been successful for facilitating neonatal screening, home testing and remote site sampling collection and transport of samples to a laboratory for analysis. In the past, a puncture site is produced on the patient to produce blood. Drops of blood are placed onto a card with a particular type of paper or other suitable absorbent material. This method is difficult for patients to use. In order to eliminate or prevent contamination, the patient can not touch the card. The protocol for taking the sample requires the patient to form a droplet large enough so that it falls onto the absorbent material. This requires a fairly large amount of blood and also is very inconvenient for the patient. If a patient happens to be squeamish about the sight of blood, the test can be that much more difficult to perform. Once the sample is taken it is shipped to a lab. In order for the lab to standardize the size of the sample, the absorbent material is punched with a hole punch in the area of the containing the dried blood spot. The punch out is the portion of the taken sample that is used for testing. There are also on-line liquid extractions devices that fix the sample size to a standard amount by passing liquid through a fixed area of the dried blood spot.

Among the disadvantages of the dried blood spot card system, are:

1) The current system requires much more blood than is often required for the analytical procedure which results in a requirement of a larger lancet gauge and ultimately more pain for the subject being tested. Standard procedures for the current system result in spots that contain approximately 80 uL of blood. For analysis methods such as DNA sequencing and LC/MS, liquid chromatography/mass spectrometry, analysis this amount of blood is orders of magnitude more than what is required and many standard protocols simply dilute the sample prior to sample analysis.
2) The current system, specifically the card format of the system, is difficult to automate for a high throughput robotic scheme. The two-dimensional nature of these cards result in inherent difficulties in grasping and manipulating these cards in a high precision manner.
3) The current system makes a multisolvent or multi stage extraction protocol very difficult to accomplish. This is especial the case when the multisolvent/multistate protocol is meant to be automated.
4) Still another disadvantage of the blood spot cards, when prepared by the punch method or online methods, is that only a subset of the entire spot is used. This is problematic since the entire blood spot area is not homogenous, as can easily be determined by simple visual inspection by noticing that the edges of the spot appear darker than the center. Chemical analysis of various regions of a single spot does result in differential chemical composition of the multitude of sample areas.
5) Still another disadvantage of the current system is that the entire area of the card is exposed to the environment and therefore the potential for contamination on the card is highly probable.
6) In the current system, the original volume of the blood sample is not known. Assumptions are required in the preparation of the sample for analysis rather than simply having a known starting volume

### BRIEF DESCRIPTION OF THE DRAWINGS

The embodiments will be readily understood by the following detailed description in conjunction with the accompanying drawings, wherein like reference numerals designate like structural elements, and in which:

FIG. 1 is a schematic view of a sample collection system, according to an example embodiment.

FIG. 2 is an exploded perspective view of a sample collection device used as part of the sample collection system, according to an example embodiment.

FIG. 3 is a perspective view of the sample collection device as assembled and ready for use, according to an example embodiment.

FIG. 4 is a bottom perspective view of the sample collection device as assembled and ready for use, according to an example embodiment.

FIG. 5 is a perspective view of the sample collection device as the absorbent material is being removed from the housing at a testing site, according to an example embodiment.

FIG. 6 is a flow diagram of a system for determining an amount of volume of blood based on an observed area of blood on the absorbent material, and analyzing the sample, according to an example embodiment.

FIG. 7 is another flow diagram of a system for determining an amount of volume of blood based on an observed area of blood on the absorbent material, and analyzing the sample, according to an example embodiment.

FIG. 8 is a schematic view of a computing system that can be used to control robots and conduct various aspects of the analyst, according to an example embodiment.

FIG. 9 shows a diagrammatic representation of a computing device for a machine in the example electronic form of a computer system, according to an example embodiment.

### DETAILED DESCRIPTION

In the following paper, numerous specific details are set forth to provide a thorough understanding of the concepts underlying the described embodiments. It will be apparent, however, to one skilled in the art that the described embodiments may be practiced without some or all of these specific details. In other instances, well known process steps have not been described in detail in order to avoid unnecessarily obscuring the underlying concepts.

FIG. 1 is a schematic view of a sample collection system 100, according to an example embodiment. The sample collection system 100 is a broad overview of how samples are collected and analyzed. The sample collection system 100 includes a laboratory 110 which includes an analysis portion 120. The system also includes a number of sites 130, 132, 134 and 136. The sites 130, 132, 134 136 can include individual doctor's offices, clinics, hospitals, households, membership gyms, retail stores, pharmacies, remote sampling sites any other facilities. At these various sites 130, 132, 134 and 136, blood samples are taken. As shown in FIG. 1, the blood samples 140, 142, 144, 146 are dried blood samples taken on a specific strip of absorbent material typically of cellulous or non-cellulous nature, and will be further described as part of this invention. The blood samples 140, 142, 144, 146 are sent to the lab or laboratory 110 where they are processed and analyzed. The analysis is conducted in the analysis portion 120. In one embodiment the analysis portion includes a liquid chromatogram/mass spectrometer system. In some embodiments the analysis and processing can be robotically controlled or otherwise automated. In other embodiments, the analysis and processing can be substantially automated or robotically controlled. In still other embodiments at least a portion of the analysis of process can be automated. Once the analysis is conducted test results are sent back to the various testing sites 130, 132, 134, 136 for discussions with patients regarding the results. Of course, the test results are correlated to the patient from whom a particular sample was taken. In one embodiment, the system 100 also includes a secure connection to the world wide web 150. A secure website hosted at the laboratory 110, or within a server on the cloud 150 can be used to transfer results to patients. A physician and patient or other health care professional can then discuss the results through one or more portals, such as a computer or computers, attached to the world wide web.

FIG. 2 is an exploded perspective view of a sample collection device 200 used as part of the sample collection system, according to an example embodiment. FIG. 3 is a perspective view of the sample collection device 200 as assembled and ready for use, according to an example embodiment. Now referring to both FIGS. 2 and 3, the sample collection device 200 will be further detailed. The sample collection device 200 includes a piece of absorbent material or absorbent material 210. In one particular embodiment the absorbent material is Whatman 903 paper available from GE Healthcare Bio-Sciences Corp. of Piscataway, N.J. 08855-1327 USA. In another embodiment, a non-cellulose material is the Agilent DMS material, 5301 Stevens Creek Blvd Santa Clara Calif. 95051 United States. As shown in FIGS. 2 and 3, absorbent material is cut to a specific size. Generally, the size the absorbent material will be uniform, in one embodiment. The sample collection device 200 also includes a handle 220, a cap 230, and a stem 240. The stem 240 attaches the absorbent material 210 to the cap 230. The handle 220 is attached to the cap 230. The stem 240 is made of a material capable of holding the absorbent material 210. The stem 240 is also made of a material that will not react or contaminate with any solvent used to extract the sample from the absorbent material 210. In one embodiment, the stem 240 is formed of stainless steel. The stem 240, in one embodiment, has the dimensions similar to a sewing needle. It should be understood that the stem can be made of other nonreactive materials, and that the dimensions of both the stem and the absorbent material 210 can be changed or different and still be within the scope of the invention.

The collection device 200 also includes a housing or clear container 250. The housing 250 includes a first open end 252 and a second open end 254. The first open end 252 is sized to allow the absorbent material 210 and the stem 240 to pass through the first opening 252 and into the main body of the housing 250. The first open end 252 captures or catches the cap 230. The cap 230 fits within the open end 252 and also acts as a stop so that the absorbent material 210 is positioned near or proximate the second open end 254. The housing 250 allows the sample device 200 to be handled without significant contamination of the absorbent material 210. In addition the housing or clear container 250 also protects the stem 240 and the absorbent material 210. The handle 220 allows for automatic processing of the sample contained within the absorbent material 210 attached to the handle 220 via the cap 230 and the stem 240. In one embodiment a robot can be used to identify the specific device 200 among many devices 200 by virtue of an identification marker or identifier, such as a barcode or QR code or similar marker, contained on the handle 220. The marker can be read by a reader. The robot includes a mechanism to grab the handle 220 and move the attached cap 230, stem 240 and absorbent material 210 to various stations where the sample on the absorbent material 210 is processed and analyzed.

In another embodiment, a barcode can be provided on the specific device at the time of manufacture. The barcode would be unique. At the time of taking or submitting the sample, the patient could be linked to the unique barcode identifier.

FIG. 4 is a bottom perspective view of the sample collection device as assembled and ready for use, according to an example embodiment. FIGS. 3 and 4 show the sample collection device 200 in an assembled form and ready for taking a sample. FIG. 4 shows the position of the absorbent material near the second opening 254 of the housing or clear container 250.

In operation, when a sample is collected or taken it is much easier on a patient. The patient's finger or other body portion is lanced to produce a source of blood for the sample. The sampling device 200 is moved into close proximity to the blood at the lance site. The absorbent material touches the blood at the Lance site, and blood is absorbed into the absorbent material 210. The sampling device is then removed and placed into a holder for a number of the sample devices. The same lance site can then be used to collect another sample with a fresh sampling device 200. In this way it is easier on the patient. The patient merely has to be pricked or lanced one time and multiple samples can be obtained from the lance site. The patient also does not have to manipulate or otherwise squeeze the area around the lance site to produce enough blood for a sample. In addition, much less blood is needed to produce a number of samples. The process is faster and more efficient than previous ways of obtaining blood samples for blood spot tests.

After a blood sample is transferred to the device 200 via the opening 254, the device 200 is placed in a holder. The absorbent paper 210 carrying the blood sample dries due to exposure to the surrounding environment. Generally, circulating air dries the blood on the absorbent material or absorbent paper. It should be noted that the blood sample is transferred to the device 200 and more specifically to the absorbent material without being touched by anyone. Therefore, the chance for contamination from such a source is minimized. In addition, the absorbent material is dimensioned to accommodate the widest blood volume range with the lowest sample dilution amount so that testing result can be accurate through a wide range of values. It has been found that an amount of blood in a sample should be in the range of 2-15 microliters. For consistency in conducting a blood test, it is desirable to substantially cover or totally cover the absorbent material. If the material is too long, too much solvent is needed to completely cover or substantially completely cover the absorbent material. Table 1 below shows dimensions which absorb 15 microliters of blood or more. The absorbent material having dimensions which absorb more than 15 microliters of blood are considered too large.

Table 2 shows the dimensions of the absorbent material where the least amount of solvent is required for a given condition where at least 2 microliters of blood are on or dried onto the absorbent material.

Table 3 shows the dimensions of the absorbent material where the sample includes at least 2-15 microliters of blood or dried blood and where the least amount of solvent is required to cover or substantially cover the absorbent material. Of course, this example is for blood or dried blood. If different materials were being tested, these dimensions could change. In other words, Table 3 shows the intersection of Tables 1 and 2.

Table 1 shows the maximum Strip capacity of Blood (micro liters) for given strip dimensions. This table only shows values that are equal to or greater than 15 uL and this is defined as “condition 1”.

TABLE 1

| | | Strip Width (mm) | | | | | | | | | | | | | | | |
|---|---|---|---|---|---|---|---|---|---|---|---|---|---|---|---|---|---|
| | | 0.5 | 1.0 | 1.5 | 2.0 | 2.5 | 3.0 | 3.5 | 4.0 | 4.5 | 5.0 | 5.5 | 6.0 | 6.5 | 7.0 | 7.5 | 8.0 |
| Strip | 1 | | | | | | | | | | | | | | | | |
| Height | 2 | | | | | | | | | | | | | | | | |
| (mm) | 3 | | | | | | | | | | | | | | | | |
| | 4 | | | | | | | | | | | | | | | | |
| | 5 | | | | | | | | | | | | | | | | 15.3 |
| | 6 | | | | | | | | | | | | | | 16.1 | 17.3 | 18.4 |
| | 7 | | | | | | | | | | | | 16.1 | 17.5 | 18.8 | 20.2 | 21.6 |
| | 8 | | | | | | | | | | 15.3 | 16.9 | 18.4 | 20 | 21.6 | 23.2 | 24.8 |
| | 9 | | | | | | | | | 15.5 | 17.3 | 19 | 20.8 | 22.6 | 24.4 | 26.2 | 27.9 |
| | 10 | | | | | | | | 15.3 | 17.3 | 19.2 | 21.2 | 23.2 | 25.2 | 27.1 | 29.1 | 31.1 |
| | 11 | | | | | | | | 16.9 | 19 | 21.2 | 23.4 | 25.6 | 27.7 | 29.9 | 32.1 | 34.3 |
| | 12 | | | | | | | 16.1 | 18.4 | 20.8 | 23.2 | 25.6 | 27.9 | 30.3 | 32.7 | 35 | 37.4 |
| | 13 | | | | | | | 17.5 | 20 | 22.6 | 25.2 | 27.7 | 30.3 | 32.9 | 35.4 | 38 | 40.6 |
| | 14 | | | | | | 16.1 | 18.8 | 21.6 | 24.4 | 27.1 | 29.9 | 32.7 | 35.4 | 38.2 | 41 | 43.7 |
| | 15 | | | | | | 17.3 | 20.2 | 23.2 | 26.2 | 29.1 | 32.1 | 35 | 38 | 41 | 43.9 | 46.9 |
| | 16 | | | | | 15.3 | 18.4 | 21.6 | 24.8 | 27.9 | 31.1 | 34.3 | 37.4 | 40.6 | 43.7 | 46.9 | 50.1 |
| | 17 | | | | | 16.3 | 19.6 | 23 | 26.4 | 29.7 | 33.1 | 36.4 | 39.8 | 43.2 | 46.5 | 49.9 | 53.2 |
| | 18 | | | | | 17.3 | 20.8 | 24.4 | 27.9 | 31.5 | 35 | 38.6 | 42.2 | 45.7 | 49.3 | 52.8 | 56.4 |
| | 19 | | | | | 18.2 | 22 | 25.8 | 29.5 | 33.3 | 37 | 40.8 | 44.5 | 48.3 | 52 | 55.8 | 59.6 |
| | 20 | | | | 15.3 | 19.2 | 23.2 | 27.1 | 31.1 | 35 | 39 | 43 | 46.9 | 50.9 | 54.8 | 58.8 | 62.7 |
| | 21 | | | | 16.1 | 20.2 | 24.4 | 28.5 | 32.7 | 36.8 | 41 | 45.1 | 49.3 | 53.4 | 57.6 | 61.7 | 65.9 |
| | 22 | | | | 16.9 | 21.2 | 25.6 | 29.9 | 34.3 | 38.6 | 43 | 47.3 | 51.7 | 56 | 60.3 | 64.7 | 69 |
| | 23 | | | | 17.7 | 22.2 | 26.7 | 31.3 | 35.8 | 40.4 | 44.9 | 49.5 | 54 | 58.6 | 63.1 | 67.7 | 72.2 |
| | 24 | | | | 18.4 | 23.2 | 27.9 | 32.7 | 37.4 | 42.2 | 46.9 | 51.7 | 56.4 | 61.1 | 65.9 | 70.6 | 75.4 |
| | 25 | | | | 19.2 | 24.2 | 29.1 | 34.1 | 39 | 43.9 | 48.9 | 53.8 | 58.8 | 63.7 | 68.7 | 73.6 | 78.5 |
| | 26 | | | | 20 | 25.2 | 30.3 | 35.4 | 40.6 | 45.7 | 50.9 | 56 | 61.1 | 66.3 | 71.4 | 76.6 | 81.7 |
| | 27 | | | 15.5 | 20.8 | 26.2 | 31.5 | 36.8 | 42.2 | 47.5 | 52.8 | 58.2 | 63.5 | 68.8 | 74.2 | 79.5 | 84.9 |
| | 28 | | | 16.1 | 21.6 | 27.1 | 32.7 | 38.2 | 43.7 | 49.3 | 54.8 | 60.3 | 65.9 | 71.4 | 77 | 82.5 | 88 |
| | 29 | | | 16.7 | 22.4 | 28.1 | 33.9 | 39.6 | 45.3 | 51.1 | 56.8 | 62.5 | 68.3 | 74 | 79.7 | 85.5 | 91.2 |
| | 30 | | | 17.3 | 23.2 | 29.1 | 35 | 41 | 46.9 | 52.8 | 58.8 | 64.7 | 70.6 | 76.6 | 82.5 | 88.4 | 94.3 |
| | 31 | | | 17.9 | 24 | 30.1 | 36.2 | 42.4 | 48.5 | 54.6 | 60.7 | 66.9 | 73 | 79.1 | 85.3 | 91.4 | 97.5 |
| | 32 | | 18.4 | 24.8 | 31.1 | 37.4 | 43.7 | 50.1 | 56.4 | 62.7 | 69 | 75.4 | 81.7 | 88 | 94.3 | 101 | |
| | 33 | | 19 | 25.6 | 32.1 | 38.6 | 45.1 | 51.7 | 58.2 | 64.7 | 71.2 | 77.7 | 84.3 | 90.8 | 97.3 | 104 | |
| | 34 | | 19.6 | 26.4 | 33.1 | 39.8 | 46.5 | 53.2 | 60 | 66.7 | 73.4 | 80.1 | 86.8 | 93.6 | 100 | 107 | |
| | 35 | | 20.2 | 27.1 | 34.1 | 41 | 47.9 | 54.8 | 61.7 | 68.7 | 75.6 | 82.5 | 89.4 | 96.3 | 103 | 110 | |
| | 36 | | 20.8 | 27.9 | 35 | 42.2 | 49.3 | 56.4 | 63.5 | 70.6 | 77.7 | 84.9 | 92 | 99.1 | 106 | 113 | |
| | 37 | | 21.4 | 28.7 | 36 | 43.4 | 50.7 | 58 | 65.3 | 72.6 | 79.9 | 87.2 | 94.5 | 102 | 109 | 117 | |
| | 38 | | 22 | 29.5 | 37 | 44.5 | 52 | 59.6 | 67.1 | 74.6 | 82.1 | 89.6 | 97.1 | 105 | 112 | 120 | |
| | 39 | | 22.6 | 30.3 | 38 | 45.7 | 53.4 | 61.1 | 68.8 | 76.6 | 84.3 | 92 | 99.7 | 107 | 115 | 123 | |
| | 40 | 15.3 | 23.2 | 31.1 | 39 | 46.9 | 54.8 | 62.7 | 70.6 | 78.5 | 86.4 | 94.3 | 102 | 110 | 118 | 126 | |
| | 41 | 15.7 | 23.8 | 31.9 | 40 | 48.1 | 56.2 | 64.3 | 72.4 | 80.5 | 88.6 | 96.7 | 105 | 113 | 121 | 129 | |
| | 42 | 16.1 | 24.4 | 32.7 | 41 | 49.3 | 57.6 | 65.9 | 74.2 | 82.5 | 90.8 | 99.1 | 107 | 116 | 124 | 132 | |
| | 43 | 16.5 | 25 | 33.5 | 42 | 50.5 | 59 | 67.5 | 76 | 84.5 | 93 | 102 | 110 | 119 | 127 | 136 | |
| | 44 | 16.9 | 25.6 | 34.3 | 43 | 51.7 | 60.3 | 69 | 77.7 | 86.4 | 95.1 | 104 | 113 | 121 | 130 | 139 | |
| | 45 | 17.3 | 26.2 | 35 | 43.9 | 52.8 | 61.7 | 70.6 | 79.5 | 88.4 | 97.3 | 106 | 115 | 124 | 133 | 142 | |
| | 46 | 17.7 | 26.7 | 35.8 | 44.9 | 54 | 63.1 | 72.2 | 81.3 | 90.4 | 99.5 | 109 | 118 | 127 | 136 | 145 | |
| | 47 | 18.1 | 27.3 | 36.6 | 45.9 | 55.2 | 64.5 | 73.8 | 83.1 | 92.4 | 102 | 111 | 120 | 130 | 139 | 148 | |
| | 48 | 18.4 | 27.9 | 37.4 | 46.9 | 56.4 | 65.9 | 75.4 | 84.9 | 94.3 | 104 | 113 | 123 | 132 | 142 | 151 | |
| | 49 | 18.8 | 28.5 | 38.2 | 47.9 | 57.6 | 67.3 | 77 | 86.6 | 96.3 | 106 | 116 | 125 | 135 | 145 | 154 | |
| | 50 | 19.2 | 29.1 | 39 | 48.9 | 58.8 | 68.7 | 78.5 | 88.4 | 98.3 | 108 | 118 | 128 | 138 | 148 | 158 | |

Table 2 shows sample dilutions at given strip dimensions assuming a min of 2 uL blood on strip and 100% of strip covered with extraction solvent. This assumes the strip is placed at the bottom of a vial and the vial has an internal dimension of the strip width plus 2 mm. The additional 2 mm allows for movement of the strip in and out of the vial. This table only shows the lowest 20% dilution results and is defined as “condition 2”.

TABLE 2

| | | Strip Width (mm) | | | | | | | | | | | | | | | |
|---|---|---|---|---|---|---|---|---|---|---|---|---|---|---|---|---|---|
| | | 0.5 | 1 | 1.5 | 2 | 2.5 | 3 | 3.5 | 4 | 4.5 | 5 | 5.5 | 6 | 6.5 | 7 | 7.5 | 8 |
| Strip | 1 | 7.5 | 10 | 12.5 | 15 | 17.5 | 20 | 22.5 | 25 | 27.5 | 30 | 32.5 | 35 | 37.5 | 40 | 42.5 | 45 |
| Height | 2 | 15 | 20 | 25 | 30 | 35 | 40 | 45 | 50 | 55 | 60 | 65 | 70 | 75 | 80 | 85 | 90 |
| (mm) | 3 | 22.5 | 30 | 37.5 | 45 | 52.5 | 60 | 67.5 | 75 | 82.5 | 90 | 97.5 | 105 | 113 | 120 | 128 | 135 |
| | 4 | 30 | 40 | 50 | 60 | 70 | 80 | 90 | 100 | 110 | 120 | 130 | 140 | 150 | 160 | 170 | 180 |
| | 5 | 37.5 | 50 | 62.5 | 75 | 87.5 | 100 | 113 | 125 | 138 | 150 | 163 | 175 | 188 | 200 | 213 | 225 |
| | 6 | 45 | 60 | 75 | 90 | 105 | 120 | 135 | 150 | 165 | 180 | 195 | 210 | 225 | 240 | 255 | 270 |
| | 7 | 52.5 | 70 | 87.5 | 105 | 123 | 140 | 158 | 175 | 193 | 210 | 228 | 245 | 263 | 280 | 298 | 315 |
| | 8 | 60 | 80 | 100 | 120 | 140 | 160 | 180 | 200 | 220 | 240 | 260 | 280 | 300 | 320 | 340 | 360 |
| | 9 | 67.5 | 90 | 113 | 135 | 158 | 180 | 203 | 225 | 248 | 270 | 293 | 315 | 338 | 360 | 383 | 405 |
| | 10 | 75 | 100 | 125 | 150 | 175 | 200 | 225 | 250 | 275 | 300 | 325 | 350 | 375 | 400 | 425 | 450 |
| | 11 | 82.5 | 110 | 138 | 165 | 193 | 220 | 248 | 275 | 303 | 330 | 358 | 385 | 413 | 440 | | |
| | 12 | 90 | 120 | 150 | 180 | 210 | 240 | 270 | 300 | 330 | 360 | 390 | 420 | 450 | | | |
| | 13 | 97.5 | 130 | 163 | 195 | 228 | 260 | 293 | 325 | 358 | 390 | 423 | 455 | | | | |
| | 14 | 105 | 140 | 175 | 210 | 245 | 280 | 315 | 350 | 385 | 420 | 455 | | | | | |
| | 15 | 113 | 150 | 188 | 225 | 263 | 300 | 338 | 375 | 413 | 450 | | | | | | |
| | 16 | 120 | 160 | 200 | 240 | 280 | 320 | 360 | 400 | 440 | | | | | | | |
| | 17 | 128 | 170 | 213 | 255 | 298 | 340 | 383 | 425 | | | | | | | | |
| | 18 | 135 | 180 | 225 | 270 | 315 | 360 | 405 | 450 | | | | | | | | |
| | 19 | 143 | 190 | 238 | 285 | 333 | 380 | 428 | | | | | | | | | |
| | 20 | 150 | 200 | 250 | 300 | 350 | 400 | 450 | | | | | | | | | |
| | 21 | 158 | 210 | 263 | 315 | 368 | 420 | | | | | | | | | | |
| | 22 | 165 | 220 | 275 | 330 | 385 | 440 | | | | | | | | | | |
| | 23 | 173 | 230 | 288 | 345 | 403 | | | | | | | | | | | |
| | 24 | 180 | 240 | 300 | 360 | 420 | | | | | | | | | | | |
| | 25 | 188 | 250 | 313 | 375 | 438 | | | | | | | | | | | |
| | 26 | 195 | 260 | 325 | 390 | 455 | | | | | | | | | | | |
| | 27 | 203 | 270 | 338 | 405 | | | | | | | | | | | | |
| | 28 | 210 | 280 | 350 | 420 | | | | | | | | | | | | |
| | 29 | 218 | 290 | 363 | 435 | | | | | | | | | | | | |
| | 30 | 225 | 300 | 375 | 450 | | | | | | | | | | | | |
| | 31 | 233 | 310 | 388 | | | | | | | | | | | | | |
| | 32 | 240 | 320 | 400 | | | | | | | | | | | | | |
| | 33 | 248 | 330 | 413 | | | | | | | | | | | | | |
| | 34 | 255 | 340 | 425 | | | | | | | | | | | | | |
| | 35 | 263 | 350 | 438 | | | | | | | | | | | | | |
| | 36 | 270 | 360 | 450 | | | | | | | | | | | | | |
| | 37 | 278 | 370 | | | | | | | | | | | | | | |
| | 38 | 285 | 380 | | | | | | | | | | | | | | |
| | 39 | 293 | 390 | | | | | | | | | | | | | | |
| | 40 | 300 | 400 | | | | | | | | | | | | | | |
| | 41 | 308 | 410 | | | | | | | | | | | | | | |
| | 42 | 315 | 420 | | | | | | | | | | | | | | |
| | 43 | 323 | 430 | | | | | | | | | | | | | | |
| | 44 | 330 | 440 | | | | | | | | | | | | | | |
| | 45 | 338 | 450 | | | | | | | | | | | | | | |
| | 46 | 345 | | | | | | | | | | | | | | | |
| | 47 | 353 | | | | | | | | | | | | | | | |
| | 48 | 360 | | | | | | | | | | | | | | | |
| | 49 | 368 | | | | | | | | | | | | | | | |
| | 50 | 375 | | | | | | | | | | | | | | | |

Table 3 shows the strip dimension combinations where “condition 1” and “condition 2” are both satisfied and where the width is greater than the height. A value of “1”, indicates where these conditions are satisfied.

TABLE 3

| | | Strip Width (mm) | | | | | | | | | | | | | | | |
|---|---|---|---|---|---|---|---|---|---|---|---|---|---|---|---|---|---|
| | | 0.5 | 1 | 1.5 | 2 | 2.5 | 3 | 3.5 | 4 | 4.5 | 5 | 5.5 | 6 | 6.5 | 7 | 7.5 | 8 |
| Strip | 1 | | | | | | | | | | | | | | | | |
| Height | 2 | | | | | | | | | | | | | | | | |
| (mm) | 3 | | | | | | | | | | | | | | | | |
| | 4 | | | | | | | | | | | | | | | | |
| | 5 | | | | | | | | | | | | | | | | 1 |
| | 6 | | | | | | | | | | | | | | 1 | 1 | 1 |
| | 7 | | | | | | | | | | | | 1 | 1 | 1 | 1 | 1 |
| | 8 | | | | | | | | | | 1 | 1 | 1 | 1 | 1 | 1 | 1 |
| | 9 | | | | | | | | | 1 | 1 | 1 | 1 | 1 | 1 | 1 | 1 |
| | 10 | | | | | | | | 1 | 1 | 1 | 1 | 1 | 1 | 1 | 1 | 1 |
| | 11 | | | | | | | | 1 | 1 | 1 | 1 | 1 | 1 | 1 | | |
| | 12 | | | | | | | 1 | 1 | 1 | 1 | 1 | 1 | 1 | | | |
| | 13 | | | | | | | 1 | 1 | 1 | 1 | 1 | 1 | | | | |
| | 14 | | | | | | 1 | 1 | 1 | 1 | 1 | 1 | | | | | |
| | 15 | | | | | | 1 | 1 | 1 | 1 | 1 | | | | | | |
| | 16 | | | | | 1 | 1 | 1 | 1 | 1 | | | | | | | |
| | 17 | | | | | 1 | 1 | 1 | 1 | | | | | | | | |
| | 18 | | | | | 1 | 1 | 1 | 1 | | | | | | | | |
| | 19 | | | | | 1 | 1 | 1 | | | | | | | | | |
| | 20 | | | | 1 | 1 | 1 | 1 | | | | | | | | | |
| | 21 | | | | 1 | 1 | 1 | | | | | | | | | | |
| | 22 | | | | 1 | 1 | 1 | | | | | | | | | | |
| | 23 | | | | 1 | 1 | | | | | | | | | | | |
| | 24 | | | | 1 | 1 | | | | | | | | | | | |
| | 25 | | | | 1 | 1 | | | | | | | | | | | |
| | 26 | | | | 1 | 1 | | | | | | | | | | | |
| | 27 | | | 1 | 1 | | | | | | | | | | | | |
| | 28 | | | 1 | 1 | | | | | | | | | | | | |
| | 29 | | | 1 | 1 | | | | | | | | | | | | |
| | 30 | | | 1 | 1 | | | | | | | | | | | | |
| | 31 | | | 1 | | | | | | | | | | | | | |
| | 32 | | | 1 | | | | | | | | | | | | | |
| | 33 | | | 1 | | | | | | | | | | | | | |
| | 34 | | | 1 | | | | | | | | | | | | | |
| | 35 | | | 1 | | | | | | | | | | | | | |
| | 36 | | | 1 | | | | | | | | | | | | | |
| | 37 | | | | | | | | | | | | | | | | |
| | 38 | | | | | | | | | | | | | | | | |
| | 39 | | | | | | | | | | | | | | | | |
| | 40 | | 1 | | | | | | | | | | | | | | |
| | 41 | | 1 | | | | | | | | | | | | | | |
| | 42 | | 1 | | | | | | | | | | | | | | |
| | 43 | | 1 | | | | | | | | | | | | | | |
| | 44 | | 1 | | | | | | | | | | | | | | |
| | 45 | | 1 | | | | | | | | | | | | | | |
| | 46 | | | | | | | | | | | | | | | | |
| | 47 | | | | | | | | | | | | | | | | |
| | 48 | | | | | | | | | | | | | | | | |
| | 49 | | | | | | | | | | | | | | | | |
| | 50 | | | | | | | | | | | | | | | | |

Once the sample is obtained in the blood sampling device 200, the blood sampling device can be shipped to a lab 110 for further processing and analysis. The blood and the blood sampling device 200 dries so this test or sampling device is easier to transport. In addition the sampling device 200 is also more durable and tough so that it can withstand shipping to the laboratory 110. It should be noted, that in some example embodiments, a cover (not shown) may be placed on the second end 252 of the container or housing 250 during transport. This would further protect the absorbent material 210 during transport and would also reduce contamination to the absorbent material 210. Furthermore a drying device such as silica gel may be contained in the cover to further aid in sample dehydration during the transportation and storage process. The drying device could also be used to keep the atmosphere within the shipping package dry during the shipping process.

FIG. 5 is a perspective view of the sample collection device as the absorbent material 210 is being removed from the housing at a laboratory 110, according to an example embodiment. More specifically the absorbent material 210, the stem 240, the cap 230 and the handle 220 are removed from the housing 250 at the laboratory 110. The handle 220 is sufficiently strong to allow a robot to pinch or grab the handle and move the sample 210. In some embodiments, it is contemplated that a robot could be configured that could grab or otherwise engage a plurality of handles 220. It should be noted that the absorbent paper 210 includes a dried blood portion 212 on the distal end of the absorbent material 210.

FIG. 6 is a schematic diagram of a system 600 for analyzing a dried blood sample, according to an example embodiment. The system 600 includes an optic system 610 and a robot 620 for grabbing the handle 220 of a sampling device 200 and positioning the dried blood portion 212 of the absorbent material 210 before the optic system 610. The system 600 also includes a processor associated memory 2000, a source of solvent 630, a container for the solvent 640 and a liquid chromatogram/mass spectrometer system 650. In one embodiment the system could include an optical reference object 215 or area standard.

The absorbent material 210 has a set of dimensions. Given the set of dimensions, the area of the dried blood portion 212 can be determined by the optic system. In one example embodiment, the optic system is a camera which takes a picture of a particular absorbent material 210. The dimensions of the dried blood portion 212 can either be determined from the picture or can be known given that the dimensions of the absorbent material 210 are also known. Once the area of the dried blood portion 212 is determined, a table or a formula within the processor(s) 2000 and its associated memory can be used to determine or correlate area to a volume of blood associated with the dried blood portion 212 of the sample 210. This determination can be made by estimating the area or otherwise determining the size of the area and relating it to a table or formula to convert the area to a volume of blood. In one embodiment, the optical reference object 215 or area standard is included in the picture with the dried blood portion 212. The area reference standard 215 can then be used in a calculation to determine the absolute area of the dried blood portion 212.

The system 600 controls the amount of solvent placed in a solvent container 640. A source of solvent 630 is controlled by the processor 2000. Once the volume of blood is determined, and amount of solvent is placed into the solvent container 640 from the source of solvent 630. The exact amount of solvent is determined by the processor 2000. The processor controls the source of solvent 630 to place an amount of solvent into the solvent container 640. The robot 620, which is also owned under control of the processor, moves the absorbent material 210 into the solvent container 640. Once the absorbent material 210 has been in the solvent for a sufficient amount of time or once the extraction is complete, the solvent with the various blood compliments from the absorbent material 210 is placed into the liquid chromatograph/mass spectrometer platform 650 for analysis. In other example embodiments the solvent containing the extracted blood components can be further processed including, mixing with other extraction solvents, dilution procedures, concentration procedures, derivatization procedures and the like, prior to being placed into the liquid chromatograph/mass spectrometer platform 650 for analysis. In one example embodiment the solvent container 640 or extraction vial, where the extraction takes place, could be replaced with a fixed cavity physically connected to the robot 620. This cavity would be of similar dimensions to the solvent container 640 and would house the solvent and absorbent material 210 during the extraction. This cavity would eliminate the need for costly and disposable extraction vials 640.

FIG. 7 is a flow diagram of a system and method 700 for determining an amount of volume of blood based on an observed area of blood on the absorbent material, and analyzing the sample, according to an example embodiment. The method 700 includes initially puncturing the finger with a lancet to produce blood 710, and placing a sampling device near the blood to transfer blood to the sample strip device 712. The sample device is then sent to a laboratory where a picture is taken of the sample strip and the dried blood portion 713. From the picture, the original volume of blood is calculated using previously determined correlation of the blood-colored area of the picture to a blood volume 714. It should be noted that in some instances a formula can be used for a given determined area. In other words, the correlation of the blood-colored area in the picture does not have to be previously determined. The method 700 also includes placing a sample strip into a tube 716 and placing an amount of solvent related to the original blood volume into the tube 718. The strip or sample strip is then removed from the tube, leaving the solvent with dissolved blood components 719. The method also includes analyzing the solvent with liquid chromatography/mass spectrometry methods 720.

FIG. 8 is a schematic view of various portions of the system 600 that further details various aspects of the analysis, according to an example embodiment. As shown in FIG. 8, two test strips 811 and 812 are shown. The two test strips have different sizes or areas of dried blood portions 811′ and 812′. A picture taken of both samples. As shown in FIG. 8, the dried blood sample is placed near a reference portion when the picture is taken. The amount of area of dried blood is determined from the picture taken.

In one embodiment, an area reference standard 815 is included in the picture with an example dried blood portion 814. This is used to in a calculation to convert a pixel count that corresponds to the dried blood portion 814 to an absolute area value.

As shown in FIG. 8 data table 820 or formula can be used to correlate the area of the dried blood sample portion 811′, 812′ to the volume of blood originally absorbed by the absorbent material. From the amount of area and the amount of volume and extraction volume for the solvent can be calculated or otherwise determined, as depicted by reference numeral 830. The samples 811, 812 are then placed in the respective extraction volumes for the samples. As shown by reference numeral 840, there is a lesser volume of solvent 841 for extraction of the components from the sample 811 then the volume of solvent 842 associated with sample 812. Basically, the amount of solvent used in each instance is proportional to the amount of dried blood on the sample 811, 812.

Now referring to 850 liquid chromatography/mass spectroscopy analysis are performed to produce a time versus intensity mapping of the total ion current. Features of the total ion current result from the solute contained in the sample solution. Of course before liquid chromatography/mass spectroscopy analysis the samples 811, 812 are removed from the sample solution 841, 842, respectively. The sample solutions 841 and 842 are what undergo liquid chromatography and mass spectroscopy analysis.

As mentioned above, the computer or processor 2000 and associated memory can be used to control many of the processes associated with analyzing the blood samples. FIG. 9 shows a diagrammatic representation of a computing device for a machine in the example electronic form of a computer system 2000, according to an example embodiment. In various example embodiments, the machine operates as a standalone device or can be connected (e.g., networked) to other machines. In a networked deployment, the machine can operate in the capacity of a server or a client machine in a server-client network environment, or as a peer machine in a peer-to-peer (or distributed) network environment. The machine can be a personal computer (PC), a tablet PC, a set-top box (STB), a Personal Digital Assistant (PDA), a cellular telephone, a portable music player (e.g., a portable hard drive audio device such as a Moving Picture Experts Group Audio Layer 3 (MP3) player, a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 2000 includes a processor or multiple processors 2002 (e.g., a central processing unit (CPU), a graphics processing unit (GPU), arithmetic logic unit or all), and a main memory 2004 and a static memory 2006, which communicate with each other via a bus 2008. The computer system 2000 can further include a video display unit 2010 (e.g., a liquid crystal display (LCD) or a cathode ray tube (CRT)). The computer system 2000 also includes an alphanumeric input device 2012 (e.g., a keyboard), a cursor control device 2014 (e.g., a mouse), a disk drive unit 2016, a signal generation device 2018 (e.g., a speaker) and a network interface device 2020.

The disk drive unit 2016 includes a computer-readable medium 2022 on which is stored one or more sets of instructions and data structures (e.g., instructions 2024) embodying or utilized by any one or more of the methodologies or functions described herein. The instructions 2024 can also reside, completely or at least partially, within the main memory 2004 and/or within the processors 2002 during execution thereof by the computer system 2000. The main memory 2004 and the processors 2002 also constitute machine-readable media.

The instructions 2024 can further be transmitted or received over a network 2026 via the network interface device 2020 utilizing any one of a number of well-known transfer protocols (e.g., Hyper Text Transfer Protocol (HTTP), CAN, Serial, or Modbus).

While the computer-readable medium 2022 is shown in an example embodiment to be a single medium, the term “computer-readable medium” should be taken to include a single medium or multiple media (e.g., a centralized or distributed database, and/or associated caches and servers) that store the one or more sets of instructions and provide the instructions in a computer readable form. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, tangible forms and signals that can be read or sensed by a computer. Such media can also include, without limitation, hard disks, floppy disks, flash memory cards, digital video disks, random access memory (RAMs), read only memory (ROMs), and the like.

A computer that executes a set of instructions is transformed into a specialized machine having a specific functional purpose.

It should be noted that the examples set forth above all deal with the sampling and processing of dried blood to test for various and assorted diseases and other maladies. It should be understood that the testing system could be used to collect and analyze other fluids or other bodily fluids for collection of samples and analysis of the same.

The embodiments were chosen and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents.

It should be noted that the sampling device 200 and a system that uses the sampling device 200 has many advantages over the DBS-cards. Among the advantages are that less blood is required for obtaining a sample or multiple samples. As a result, a smaller lancet that makes a smaller wound can be used. This translates to less pain for test subject or patient. The sample device can be handled by a robot. In one example, a robot can handle the sample via the handle and stem of the sample device. As a result, sample preparation can be more easily automated. The sample device or a system that uses a plurality of sample devices is also more amenable to complex sample preparation schemes, such as those that require multiple extraction solvents and/or multiple stages. In addition, when using the sampling device 200, the entire sample is used in the extraction. This avoids the problem of sampling a subset of heterogeneous blood spot contained on the DBS-card. Yet another advantage is that the sampling device 200 is far less prone to contamination during the sample collection, shipment and sample preparation procedures. Still another advantage is that the sampling device 200 allows for direct determination of the original blood sample volume which can then be used for subsequent analytical calculations.

While the embodiments have been described in terms of several particular embodiments, there are alterations, permutations, and equivalents, which fall within the scope of these general concepts. It should also be noted that there are many alternative ways of implementing the methods and apparatuses of the present embodiments. It is therefore intended that the following appended claims be interpreted as including all such alterations, permutations, and equivalents as fall within the true spirit and scope of the described embodiments.

What is claimed:

1. A blood collection system comprising:
a housing having at least one open end; and
a absorbent material positioned within the housing, the absorbent material having at least one end positioned proximate the at least one open end of the housing.

2. The body fluid collection system of claim 1 wherein the housing is made of a material which is contaminant-free with respect to a fluid to be collected on the sample absorbent material.

3. The body fluid collection system of claim 1 wherein the housing is adapted to be moved by a robotic mechanism.

4. The body fluid collection system of claim 1 wherein the housing has a second opening therein which is on an opposite end of the housing, the fluid collection system further comprising:
a cap which covers the second opening in the housing; and
a stem attached to the cap and attached to the absorbent material.

5. The body fluid collection system of claim 4 wherein the length of the stem is selected to hold at least one portion of the absorbent material near at least one open end go the housing.

6. The body fluid collection system of claim 4 wherein the stem is made from a material that is contaminant-free with respect to the sample and the sample absorbent material.

7. The body fluid collection system of claim 4 wherein the stem is made from a nonreactable material with respect to solvents and other fluids used to test the sample.

8. The body fluid collection system of claim 4 wherein the stem is made from a material that is contaminant-free with respect to the sample and the sample absorbent material.

9. The body fluid collection system of claim 4 wherein the stem is made from stainless steel.

10. The body fluid collection system of claim 4 wherein the stem is adapted for handling by a robotic system.

11. The body fluid collection system of claim 4 wherein the absorbent material is dimensioned to facilitate the widest blood volume range with the lowest sample dilution possible.

12. The body fluid collection system of claim 4 wherein the cap and the absorbent material are removable from the housing.

13. A method of collecting blood samples comprising:
positioning a absorbent material near a source of blood;
holding the absorbent material with a housing, the absorbent material absorbing a volume of blood;
moving the housing and absorbent material to a position where the absorbent material can dry.

14. The method of claim 13 further comprising shipping the housing and the absorbent material to a lab location.

15. The method of claim 13 wherein holding the sample with the housing includes:
attaching the absorbent material to a stem; and
attaching a stem to the housing, the length of the stem selected to position a portion of the absorbent material near an opening in the housing.

16. The method of claim 13 wherein holding the sample with the housing includes:
attaching the absorbent material to a stem; and
attaching a stem to a cap positioned over a first opening in the housing, the length of the stem selected to position a portion of the absorbent material near a second opening in the housing.

17. The method of claim 15 further comprising labeling the stem with an identifier.

18. The method of claim 15 further comprising:
removing the stem and absorbent material from the housing; and
moving the stem and absorbent material using a robot, the stem and absorbent material moved to at least one different position to complete a test of a dried blood portion on the absorbent material.

19. A method of determining volume of a dried blood sample on an absorbent material comprising:
determining an area of the blood on the absorbent material;
equating the area of blood to a volume of blood sampled.

20. The method of claim 19 wherein determining an area of blood includes referring to a lookup table that relates the area of blood on a absorbent material to the volume of blood on the absorbent material.
